(19) United States
(12) Patent Application Publication
(10) Pub. No.: US 2011/0236333 A1
(43) Pub. Date: Sep. 29, 2011

(54) Non-lonic surfactant solubilized high catechin content emulsion

(75) Inventor: William Edwin Driessen, Minneapolis, MN (US)

(73) Assignee: William Edwin Driessen, Minneapolis, MN (US)

(21) Appl. No.: 12/932,265

(22) Filed: Feb. 22, 2011

Related U.S. Application Data
(60) Provisional application No. 61/308,336, filed on Feb. 26, 2010.

(51) Int. Cl. A61K 8/49; A61Q 15/00

(52) U.S. Cl. 424/65

(57) ABSTRACT

The present invention solves the problem that skin or coat treatment products which use catechin extract in a “cat-ionic” or “an-ionic” base have tended to be unstable. The problem addressed is the precipitation and color change (darkening) in just a few hours/days where products would not be commercially viable due to extremely short shelf life. The Non-Ionic Surfactant Solubilized High Catechin Content Emulsion invention achieves an efficacious level of catechins (in ranges up to and over 2% by weight) as a proper emulsification method for the high catechin content in a skin, scalp, or coat treatment that does not separate or darken and has a much longer shelf-life.

### RELATED APPLICATIONS

Priority is claimed in this application to the Provisional Patent Application No. 61/308,336, accorded a filing date of Feb. 26, 2010, by same inventor.

### BACKGROUND OF THE INVENTION

Humans and all other mammals (hereafter “Animal”(s)) are living biological organisms which tend to secret various substances. Bromhidrosis (or “body odor”) is the smell of the eco-environment created within these secretions. Once the body, skin, scalp, or coat is covered in secretions they are usually accompanied by bacteria or other microbial substances growing in the microscopic spaces.

The secretions themselves are not only harmless to Animals; they are also needed and desirable for a normal and healthy skin, scalp, or coat. It is the unwanted bacteria and other potential microbial infestations that are the actual causes of odors. This invention uses the powerful antimicrobial properties of green tea catechins (e.g. Natural Catchin Extract from Green Tea, hereafter “Extract”) combined with an effective surfactant (e.g. Polyglyceryl Monolaurate, or Polyglyceryl-10 Monolaurate, or Decaglyceryl Monolaurate, hereafter “Surfactant”)

Catechins (not the green tea itself) are the specific ingredient having the effective antimicrobial attributes. Combined with the gentle cleansing and extra anti-microbial action of the non-ionic Surfactant, the combination can effectively be applied. The higher catechin concentrations offer benefits, among other things, of killing and/or preventing the bacteria from growing—and it can deodorize the skin and hair—but it is not as harsh on the skin of the Animal. The catechins can also kill (or more accurately “deactivate”) enzymes that live in the scalp and are detrimental to hair health. Deactivation of these enzymes can lead to generally improved status of scalp health, as measured by such factors as Hair Folicles/sq. cm, stiffness of hair follicle, and average diameter of hair follicles.

The non-ionic Surfactant has high foaming and cleaning properties, but washes away less amino acids from the skin compared to other common skin, scalp, or animal coat cleaning and/or treatment product bases.

It is not new to include “green tea” in a skin or coat treatment product. However, catechins as an “active” ingredient require higher concentration than those currently available. Without the high catechin content, products will not have the same effect as the present invention and some may consider the “Green Tea” product branding only a marketing gimmick.

### BRIEF SUMMARY OF THE INVENTION

A problem in skin or coat treatment products that use a “cat-ionic” or “an-ionic” base has been instability. There will be precipitation and color change (darkening) in just a few hours/days and the product would not be commercially viable due to extremely short shelf life. An objective of the present invention is to achieve an efficacious level of catechins (in ranges up to and over 2% by weight) a proper emulsification method for the high catechin content in a skin, scalp, or coat treatment is one embodiment of the invention. In other embodiments of the invention the objective might be to deodorize rugs or carpets or even for human use such as a deodorizing shampoo. In yet other embodiments, the objective may not be to deodorize (or just to deodorize); there are numerous benefits or healthy consequences that can be achieved by applying or otherwise presenting a high catechin content emulsion to a system or living organism. The benefits of green tea, in particular, have been well documented.

This invention solves the instability problem by using a non-ionic Surfactant (e.g. Polyglyceryl Monolaurate). It is stable with green tea catechins at high levels and will not easily precipitate nor easily change (darken) color. It is common that bacteria living on the surface of the scalp of Animals produce waste products that cause odors. Commonly available shampoos or skin treatments wash away oils and odor, but do not kill the microorganisms that cause the odor. Immediately after shampooing the animal with currently available products, the bacteria begins to multiply and the odor quickly returns.

Other commonly available “deodorizing” or “treatment” shampoos use chemicals to kill the bacteria, but there is a harsh effect to the Animal's skin. Dryness occurs from washing away amino acids from the skin surface and can result in dandruff. The dander welcomes more unwanted microbial pests that feed on it.

Extract is a (food grade) natural catechin extract from green tea It is an herbal extract, therefore it is not designed to do anything in particular. The surfactant is nonionic, which means for this embodiment, the Extract emulsifies well in the Surfactant and results in, among other things, having a superior detergent and foaming ability. Since the “surfactant” is non-ionic, and has superior detergency and foaming ability. The “extract” has superior “deodorizing” and “anti-microbial” activity. The “surfactant” in this embodiment acts as the delivery vehicle.

### Surfactant

Nonionic surfactants comprise a broad group of over 250 types of surface-active molecules. They can include fatty acid amides and esters, alcohol amine, and phenol alkoxylates, fluorinated alcohols, organic polymers, and organo-silicone copolymers. The international trade Commission Reported in 1976 that Nonionic Surface-Active compounds produced in the United States as follows: Carboxylic acid amides; Carboxylic acid esters; Alkylphenol ethoxylates; and Nonbenzenoid ethers.

Applicant would contemplate that many surfactants exists and not all have been tried, but the Applicant of this invention has discovered that a surfactant such as e.g. Polyglyceryl Monolaurate. In specific embodiments, it is more accurately called either Polyglycerly-10 Monolaurate, or Decaglyceryl Monolaurate); it is a nonionic surfactant, which conforms to the Japanese Cosmetic Ingredients Codex and the Japanese Standard of Food Additives. This Surfactant, then, is a superior detergent with excellent foaming ability. Unlike conventional polyglyceryl fatty acid esters, Surfactant has a monoester as its main ingredient. It has a very good low irritant composition which is mild on the eyes and skin. Surfactant will not lead to dry or tight skin and tends to leave no slimy residue on the skin. During application and/or cleansing Surfactant has an appropriate wet feeling on the skin. It is stable under a broad pH range and thanks to all the above described qualities Surfactant can be effectively used in face cleansers, body soap, shampoo and hand soap.

Surfactant Chemical and Physical Properties in a Preferred Embodiment
Appearance: Light yellowish liquid with a specific odor
Saponification Value: 30-45
Loss on Drying: 48-52
Residue on Ignition: Less than 0.5%
Arsenic as AS2O3: Less than 2 ppm
Heavy Metals as Pb: Less than 20 ppm
pH: 3.7-4.9
   Storage Recommendations: Surfactant should be stored cool and dry conditions.
   Packaging: Surfactants are often available as iron drums of 180 kg net.

In the preferred embodiment of a skin, hair, or coat cleanser, “High Foaming” “Food-Grade” “nonionic surfactant” was found superior. Applicant also considers that many nonionic surfactants do not produce foam (some are actually used as anti-foaming agents for industrial applications). These would be preferred in non-foaming applications such as creams, oils, or other than the Animal applications. The term “Food-Grade” will also reduce the number of possible compounds. Many nonionic surfactants are used in herbicides, lubricants, or other industrial applications.

The Polyglycerol Monolaurate is commercially available and is primarily in the form of Polyglycerol approx 10%, Mono-Ester approx 78%, and Di- and higher esters approx 12%). It is typically mixed with equal amounts of water before incorporation into the final product. Other concentrations of premixing are still effective in combination with other concentrations of the Extract.

### Extract

Extract is a commercially available high catechin content natural green tea extract which has been specifically supplied for the making of Human and or Animal care products. Catechin content has been shown to be greater than 70%.

Green Tea Catechins are a subset of compounds known as “Flavonoids.” Catechins are found in green tea, and also known as a type of “polyphenols” because they have several hydroxyl (OH) groups in each molecule. Catechins are considered the main healthy anti-oxidant component present in green tea. The extraction process normally involves extraction with water, purification, decaffeination, and condensation, where it is sprayed, dried, and collected as an embodiment of the Extract.

Extract Chemical and Physical Data in a Preferred Embodiment
Appearance: Light yellow-brown powder
Solubility: Water Soluble
Total Polyphenols: >90% (Colorimetric method) (50-98% in other embodiments)
Total Catechins: >70% (HPLC) (40-98% in other embodiments)
EGCg: >40% (HPLC) (20-60% in other embodiments)
Caffeine: <1% (HPLC) (may be higher in other embodiments).
Theobromine: <0.5% (HPLC)(may be higher in other embodiments)
Loss on Drying: <6% (105° C., 3 hour) (may be higher in other embodiments)
Other: <1% (typical)
(Note: Many ingredients, active ingredients, or trace elements may be found in “high catechin content” extracts)

The primary source of catechins is Green Tea (camellia sinensis). However catechins also exist in a wide variety of other plants, including Cocoa (and seeds from Theobroma cacao) and Mimosa catechu (Acacia catechu). Catechins are also found in many other plant species, for example fruits, vegetables, and wine. Additionally, it is possible to synthesize catechins, but to date and for the foreseeable future, the synthetic material is not a viable option due to high production costs.

### DESCRIPTION OF THE INVENTION AND DRAWINGS

FIG. 1 shows that Green Tea Catechins have strong deodorant activity, they readily react with compounds that are known causes of bad odors, for example, methyl-mercaptan. Epigallocatechin gallate (EGCg) is a major compound in green tea polyphenols. Methyl Mercaptan (CH₃SH) can be a contributor to various bad odors, for example, Methyl Mercaptan is a main contributing factor to halitosis. The lower case “g” of the “EGCg” designation indicates that there is a “gallate” portion of the Epigallocatechin gallate, which is the most abundant catechin derivative found in green tea. Other derivatives with or without the gallate portions are contemplated by this disclosure.

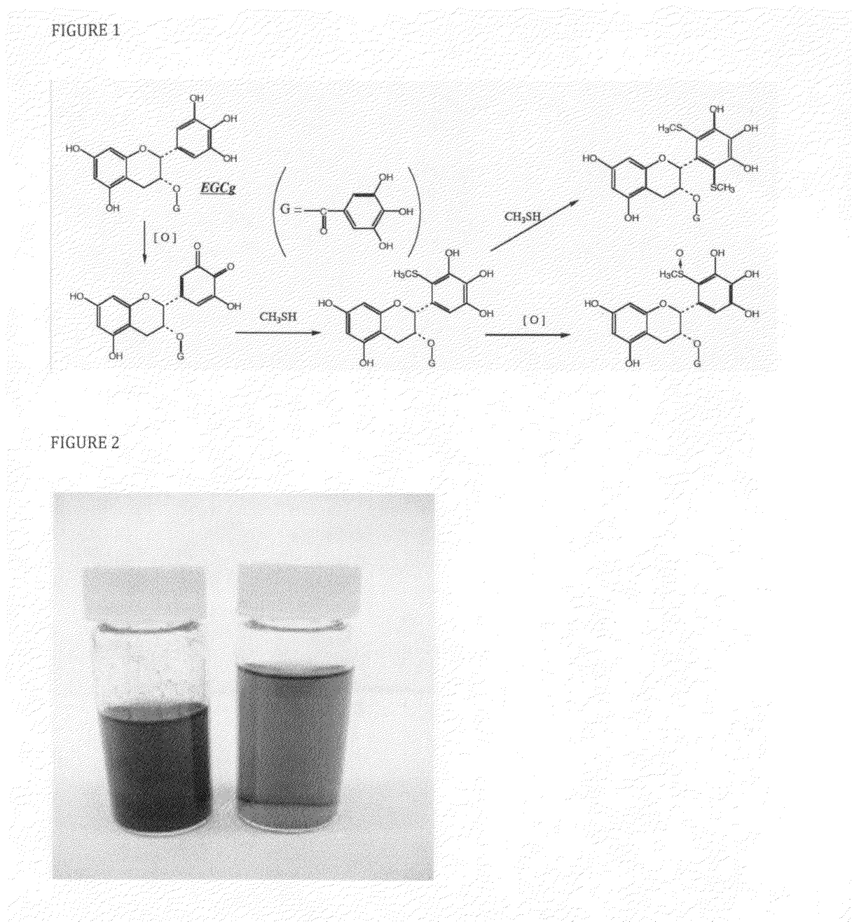

FIG. 2 is best shown by photograph rather than ink drawing, because photographs are the only practicable medium for illustrating the claimed invention. The picture in FIG. 2 shows how this invention solves the “stability” problem. The addition of Green Tea Catechins to regular shampoos can cause precipitation. The incompatibility also causes a reduction in viscosity and reduced foaming of the shampoo. The surfactant's action in the presence of the ECG (or gallate in the ECGg) creates the first known Non-Ionic Surfactant Solubilized High Catechin Content Emulsion.

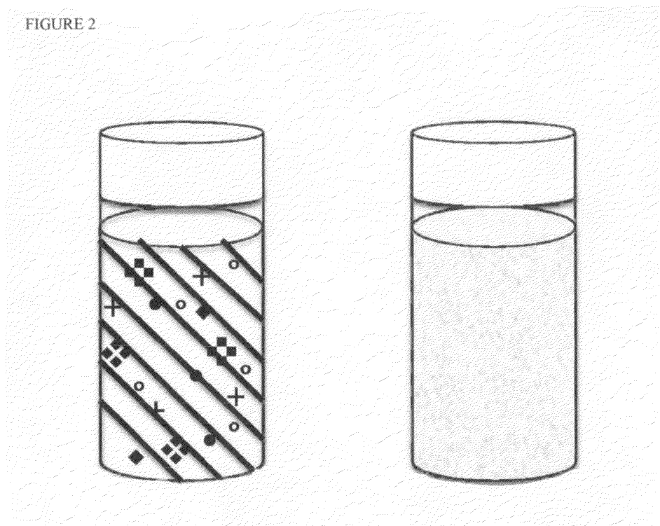

FIG. 3 is a chart showing results of green tea Catechins exhibiting good deodorant effect against ammonia. A 25% ammonia solution, incubated at 37° C. in a glass vial, with collection of “head-space” air injected into the vial, is added to a Plastic bottle containing a cotton ball previously soaked with green tea catechins and then incubated for an additional 5 minutes. 1 ml of head-space are is then taken and analyzed by an ammonia gas detector system (Gastec Corp. Japan).

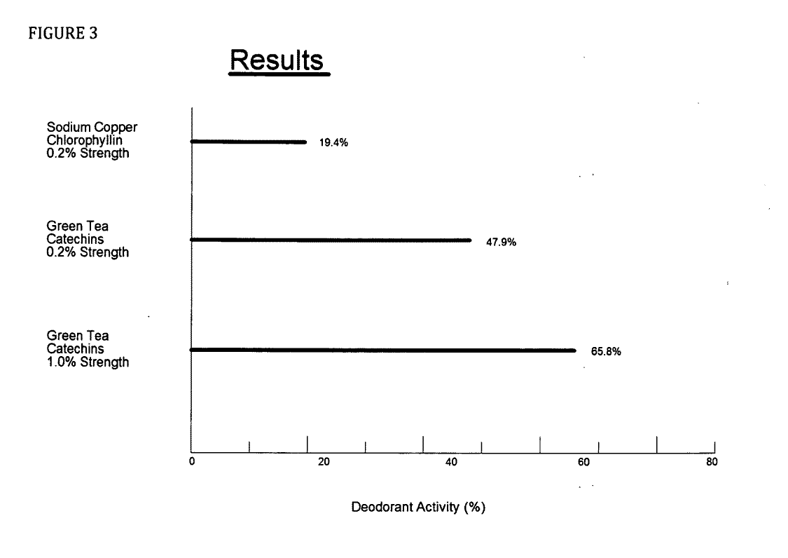

FIG. 4 is a chart showing results of green tea Catechins exhibiting good deodorant effects against methyl mercaptan (MMC) pertaining to several other deodorizing compounds. The test method included adding the deodorant liquid to a glass vial at a concentration of 300 ppm, then adding a phosphate buffer and the methyl mercaptan at 10 ppm EtOH solution (or approximately 30 μl) incubating at 37° C. for 15 minutes, and then measuring the methyl mercaptan present in the head-space using a gas-chromatograph.

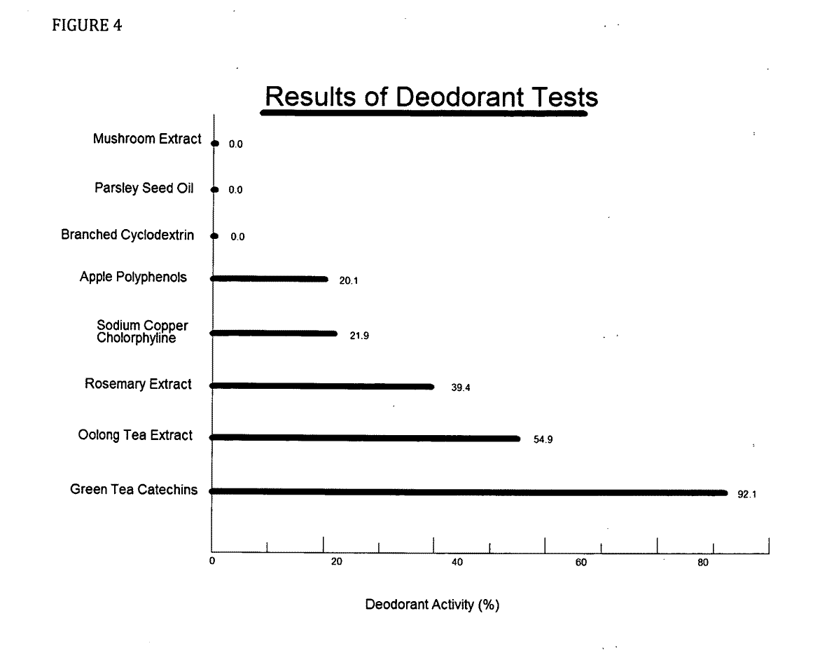

1. Water based stable composition comprising about greater than 2% catechins with a surfactant.

2. The composition in claim 1 wherein said surfactant is a Polyglycerol Monolaurate.

3. The composition in claim 1 wherein said catechins are extracted from green tea.

4. The composition in claim 1 wherein said about greater than 2% catechins with a surfactant are contained in a shampoo.

5. The composition in claim 4 wherein said shampoo is a pet shampoo.

6. The composition in claim 4 wherein said shampoo is a human shampoo.

7. The composition in claim 1 wherein said about greater than 2% catechins with a surfactant are contained in a skin, scalp, or coat application product.

8. The composition in claim 1 wherein said composition is an emulsion.

9. The composition in claim 1 wherein said surfactant is a non-ionic surfactant.

10. The composition in claim 1 wherein said about greater than 2% catechins is 1.6%

11. The composition in claim 1 wherein said about greater than 2% catechins is 1.7%

12. The composition is claim 1 wherein said about greater than 2% catechins is between 1.5% and 2%.

13. The composition in claim 1 wherein said about greater than 2% catechins is greater than 2%.

14. The composition is claim 1 wherein said about greater than 2% catechins is 1.8%

15. The composition is claim 1 wherein said about greater than 2% catechins is 1.9%

16. Method of deodorizing treatment for humans or animals comprising:
mixing into a stable mixture, an amount about greater than 2% of catechins into a water soluble emulsion with a surfactant; and applying said soluble emulsion to an animal or human.

17. Method making a deodorizing treatment for humans or animals comprising:
mixing into a stable mixture, an amount about greater than 2% catechins into a water soluble emulsion with a surfactant; and bottling or otherwise packaging for human or animal use.
